(19) United States
(12) Patent Application Publication
(10) Pub. No.: US 2007/0197988 A1
(43) Pub. Date: Aug. 23, 2007

(54) CPM pad with fiber filling

(75) Inventors: Ross P. Bieling, Sanford, FL (US); Alan C. Bieling, Seminole, FL (US)

Correspondence Address: MCKEE, VOORHEES & SEASE, P.L.C., 801 GRAND AVENUE, SUITE 3200, DES MOINES, IA, 50309-2721, US

(73) Assignee: Select Medical Products, Inc., Pinellas Park, FL (US)

(21) Appl. No.: 11/360,206

(22) Filed: Feb. 23, 2006

(51) Int. Cl. A61F 13/15

(52) U.S. Cl. 604/367; 602/41

(57) ABSTRACT

A patient pad for a continuous passive motion machine is provided with an inner fabric layer of wicking material, an outer fabric layer, and a fiber fill layer sandwiched between the inner and outer fabric layers. Quilting ties the fabric layers and fiber fill layer together. The fiber fill layer is pleated in an accordion arrangement to provide for resilient compressibility. The fiber material provides aeration and breathability for patient comfort.

### BACKGROUND OF THE INVENTION

CPM pads are well known in the art, and typically include an internal foam layer covered with fabric material. CPM pads are also known with a fleece covering of either natural or synthetic material. The pads are normally attached to the CPM machine to protect and cushion the patient's limb from direct contact with the machine.

The foam cushion in the pad may be either open or closed cell. Closed cell foam has a higher density than open cell foam. Closed cell foam has virtually no breathability, while open cell foam has limited breathability. Thus, a foam pad increases patient's sweating, which is undesirable.

Furthermore, foam is a petroleum-based product, which has seen significant price increases in recent years as oil prices have risen. Manufacturers have tried to offset the rising price by reducing the thickness of the foam, such that the pads have become thinner, thereby affording less cushioning to the patient.

Natural and synthetic quilt batting is also known in other fields of art, such as use in apparel, footwear, home furnishings, needlecraft, furniture, and sleep products. Typical products in these markets include cushion and craft fibers, pillows, mattress padding, garment insulation, active wear, duvets, sleeping bags, and upholstery. Fiber products are also used in industrial applications, such as substrates or coated products, thermal insulation, sound insulation, liquid and air filtration, safety products, medical patient care, automotive insulation, and filtration products. Such uses of fibers normally are not concerned with both padding and breathability of the product. Thus, such uses are irrelevant to a CPM pad which requires both cushioning and breathability for a patient's comfort.

Accordingly, a primary objective of the present invention is the provision of an improved CPM pad having a fiber filling.

Another objective of the present invention is the provision of a CPM pad having an accordion style fabric fill to provide enhanced cushioning for a patient's limb.

A further objective of the present invention is the provision of an improved patient pad for use with medical treatment therapy machines having a batting layer sandwiched between two fabric layers, with at least one of the fabric layers being a wicking material to wick away moisture from the patient's skin.

Still another objective of the present invention is the provision of an improved patient medical pad having enhanced aeration and breathability.

Another objective of the present invention is the provision of a thicker patient pad to provide increased padding and comfort for the patient.

Yet another objective of the present invention is the provision of an improved patient pad which is economical to manufacture, and durable and safe in use.

These and other objectives will become apparent from the following description of the invention.

### BRIEF SUMMARY OF THE INVENTION

The patient medical pad of the present invention is intended for use with continuous passive motion machines and other medical therapy machines. The pad includes an inner fabric made of a wicking material and an outer fabric. A fiber fill is sandwiched between the inner and out fabrics. Quilting ties the fabrics and fiber fill together and prevents shifting of the fiber fill between the fabric layers.

The fiber fill may be a synthetic or natural material and is arranged in an accordion fashion, with the pleats being transversed to the fabric layers. Preferably, the fiber fill is woven, rather than spun. The fiber fill, or batting, provides substantial aeration and breathability so as to minimize patient's sweating. The wicking fabric removes moisture from the patient's skin. The fabric fill is resilient, and does not remain compacted or flattened after use.

### BRIEF DESCRIPTION OF THE DRAWINGS

FIG. 1 is a perspective view of a continuous passive motion machine having pads according to the present invention.

FIG. 2 is an elevation view of a CPM pad according to the present invention.

FIG. 3 is a sectional view of the pad taken along lines 2-2 of FIG. 2.

### DETAILED DESCRIPTION OF THE PREFERRED EMBODIMENT

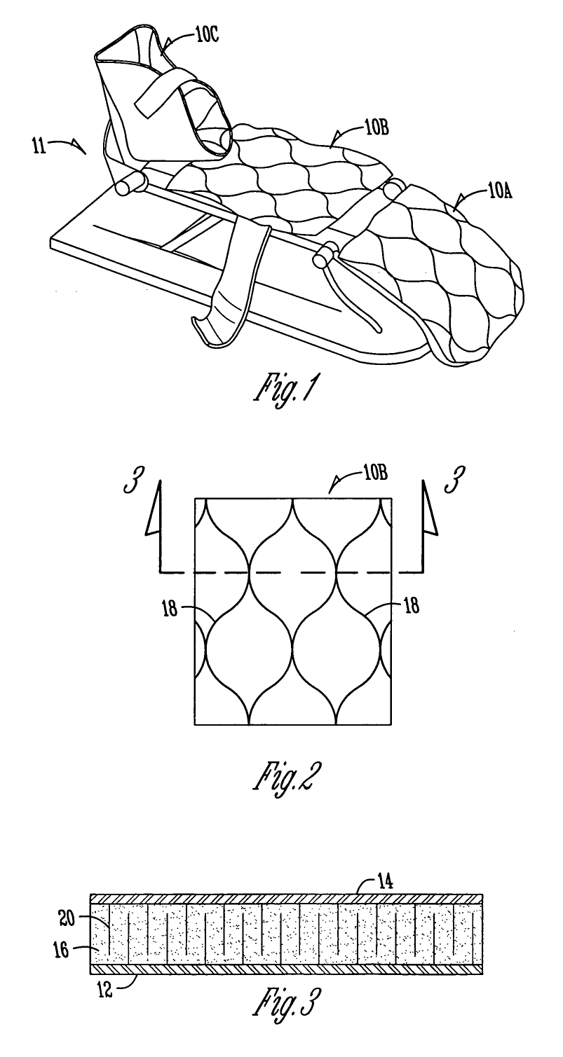

FIG. 1 shows pads 10A, 10B and 10C for use on a continuous passive motion (CPM) knee machine 11 to protect a patient's leg. Other types of padded CPM machines for use with a patient's arm or hand are also known and contemplated within the scope of the present invention.

FIG. 2 shows one embodiment of the medical therapy thigh pad 10A according to the present invention which is particularly adapted for use with the (CPM) machine 11 of FIG. 1. As seen in FIG. 3, the pad 10A has an inner fabric layer 12, an outer fabric layer 14, and an internal fiber layer 16 sandwiched between the fabric layers 12, 14. Preferably, the inner fabric layer 12 is made of a wicking material so as to carry moisture away from a patient's skin. The pad 10A is quilted, with stitching 18 having any desired pattern. The quilting ties the fabric layers 12, 14 and fiber layer 16 together and prevents movement of the fiber material 16 within the fabric layers 12, 14. Other than shape, the pads 10B and 10C have the same construction as the pad 10A.

The fiber layer 16 is a batting made of natural or synthetic material. Preferably, the fiber fill layer 16 is made of woven fibers, as opposed to spun fibers. The fiber fill layer 16 is pleated in an accordion fashion, as seen in FIG. 3, with the pleats 20 extending perpendicular to the fabric layers 12, 14. Thus, the fiber layer 16 has substantial breathability for patient comfort. Also, the fiber layer 16 provides cushioning or padding for the patient's limb. The pleats 20 in the fiber layer 16 allow for resilient compressibility of the pad 10.

Use of the fiber batting or fill provides better aeration and breathability as compared to foam filled pads, and thus increases patient comfort. The wicking material of the inner fabric layer 12 also provides increased patient comfort, as compared to fleece lined pads, due to improved moisture wicking capabilities.

It is contemplated that fiber-filled pads similar in construction to pads 10A, 10B and 10C can be used with other medical therapy machines to protect various parts of a patient's body.

The invention has been shown and described above with the preferred embodiments, and it is understood that many modifications, substitutions, and additions may be made which are within the intended spirit and scope of the invention. From the foregoing, it can be seen that the present invention accomplishes at least all of its stated objectives.

What is claimed is:

1. A continuous passive motion machine pad, comprising:
an inner fabric of wicking material;
an outer fabric;
a fiber fill between the inner and outer fabrics; and
quilting to tie the fabrics and fiber fill together.

2. The pad of claim 1 wherein the fiber fill is folded in an accordion arrangement.

3. The pad of claim 1 wherein the fiber fill is breathable.

4. The pad of claim 1 wherein the fiber fill is woven.

5. The pad of claim 1 wherein the fiber fill has greater breathability than foam.

6. An improved patient pad for use with medical treatment therapy machines, comprising:
a batting layer sandwiched between two fabric layers;
one of the fabric layers being a wicking material to wick moisture away from a patient's skin; and
the batting being folded in an accordion manner with pleats extending substantially perpendicular to the fabric layers.

7. The improved patient pad of claim 6 further comprising quilting to tie the fabric layers and batting together.

8. The improved patient pad of claim 6 wherein the batting is a woven fiber.

9. The improved patient pad of claim 6 wherein the batting is breathable.

10. The improved patient pad of claim 6 wherein the breathability is greater than that of foam.

11. The improved patient pad of claim 6 wherein the batting is selected from a group comprised of synthetic or natural materials.

12. The improved patient pad of claim 6 wherein the batting is resilient.

13. A medical therapy pad for patient comfort, comprising:
an inner fabric layer of wicking material;
an outer fabric layer;
a pleated fiber layer between the fabric layers to provide cushioning and breathability for a patient using the pad.

14. The medical therapy pad of claim 13 wherein the pleats are transverse to the fabric layers.

15. The medical therapy pad of claim 13 wherein the fiber layer is a woven material.

16. The medical therapy pad of claim 13 wherein the fiber layer is a synthetic or natural.

17. The medical therapy pad of claim 13 further comprising quilting to tie the fabric and fiber layers together.

18. The medical therapy pad of claim 13 wherein the fiber layer is resilient.
